(19) United States
(12) Patent Application Publication
(10) Pub. No.: US 2018/0216124 A1
(43) Pub. Date: Aug. 2, 2018

(54) METHOD OF PRODUCING PLASTID TRANSFORMANTS

(71) Applicant: HONDA MOTOR CO., LTD., MINATO-KU, TOKYO (JP)

(72) Inventors: Asuka YAMAGUCHI, TOKYO (JP); Jiro OKUMA, WAKO-SHI (JP); Yasuhiro KONDO, KAWAGOE-SHI (JP); Yoshitsugu HIROSE, WAKO-SHI (JP)

(21) Appl. No.: 15/510,377

(22) PCT Filed: Aug. 24, 2016
(86) PCT No.: PCT/JP2016/075375

§ 371 Date: Mar. 10, 2017

(30) Foreign Application Priority Data
Aug. 28, 2015 (JP) 2015-169817
Mar. 17, 2016 (JP) 2016-054448

(51) Int. Cl. C12N 15/82; C07K 14/415

(52) U.S. Cl. CPC C12N 15/8214; C12N 15/8241; C07K 14/415; C12N 15/821

(57) ABSTRACT

A method of producing plastid transformants including a process (a) in which genes coding for proteins having a function of neutralizing nitrite toxicity are introduced into plant tissues and a process (b) in which the plant tissues into which the genes are inserted in the process (a) are cultured in a medium containing nitrite nitrogen having a concentration at which growth of wild type plant cells is suppressed or a concentration at which regeneration of wild type plants is suppressed and thus plastid transformants obtained by inserting the genes into plastid genomes are screened.

### TECHNICAL FIELD

The present invention relates to a method of producing plastid transformants, and more specifically, to a method of producing plastid transformants in which exogenous genes are introduced into plant tissues having differentiated plastids, and genes coding for proteins that neutralize nitrite toxicity such as nitrite reductase genes are used as selectable marker genes to screen transformants.

Priority is claimed on Japanese Patent Application No. 2015-169817, filed Aug. 28, 2015, and Japanese Patent Application No. 2016-054448, filed Mar. 17, 2016, the content of which is incorporated herein by reference.

### BACKGROUND ART

The plastid (chromogen) of plants is an organelle containing pigments in plant cells, has its own genome, and performs self-growth. The chloroplast, which is a type of plastid, is the most distinguishing characteristic of plant cells, and performs photosynthesis, carbon, nitrogen and sulfur assimilation and the like. The chloroplast contains genes coding for proteins of about 50% of total soluble proteins in chloroplasts in the genome, and has a very high protein synthesis ability. In particular, in higher plants, it is known that there are a plurality of chloroplasts in cells and the number of chloroplast genomes is 5,000 to 10,000 as many as the nuclear genome. When exogenous genes are introduced into chloroplasts that are present at a great amount in plants, since mass production of proteins that the exogenous genes code is possible in plants, a plastid transformation technique is being focused as an inexpensive protein producing technique.

Plastid transformation in higher plants is basically accomplished by inserting a vector designed to interpose genes of interest and drug selectable marker genes between sequences homologous to plastid genomes. The vector is introduced into plant cells by particle bombardment, cells including plastid genomes that are transformed by homologous recombination are then repeatedly cultured on a medium (a screening medium) containing a selectable drug, and cells including transformed plastids are screened. In this process, a cell population in a (heteroplasmic) state in which wild type plastids and transformed plastids are mixed is changed to a (homoplasmic) state in which all cells retain transformed plastids, plants are regenerated from screened homoplasmic transformation cells and thus plastid transformation plants are obtained. That is, a plastid transformation method mainly includes a transformation process in which exogenous genes are inserted into plastid genomes and a process in which cells and plants containing transformed plastid genomes are screened.

Plastid transformation in higher plants according to such a method has been attempted in a large number of plants so far. However, a stable transformation system is established only in tobacco that is a dicotyledonous plant. In a plastid transformation method of tobacco, in general, introduction into a piece of leaf tissues is performed using a regeneration system from chloroplasts. Screening of transformed plastids after introduction is performed by spectinomycin resistance screening (refer to Non-Patent Document 1) using spectinomycin/streptomycin inactivated genes (aminoglycoside-3′-adenyltransferase; aadA) as a selectable marker, kanamycin resistance screening (refer to Non-Patent Documents 2 and 3) using kanamycin resistance genes as a selectable marker, the herbicide phosphinothricin (PPT) resistance screening (refer to Non-Patent Document 4) using bar genes, or the like.

On the other hand, in monocot plants, a screening method that is successfully used in dicotyledonous plants has been attempted to prepare plastid transformants. For example, it has been reported that plastid transformation in a callus (a dedifferentiated cell group) of rice is performed according to aadA genes/streptomycin screening or bar genes/PPT screening that are the same as in tobacco (refer to Non-Patent Documents 5, 6, and 7). However, in all reports, prepared products are heteroplasmic transformants, and homoplasmic transformants are not obtained. Furthermore, preparation efficiency of transformants is low. While there is another report including the fact that plastid transformants can be prepared by aadA gene screening using multiple shoots of corn as an introduction material, details of a specific method is unknown (refer to Patent Document 1).

In this manner, plastid transformation in monocot plants is difficult. A main factor thereof is considered due to the fact that a successful screening method in dicotyledonous plants is not successful for screening monocot plants. Since drug sensitivity and a response system in cells are different between species, it is necessary to modify a screening method of the related art or develop a new screening system according to a target species. Actually, since monocot plants have resistance to spectinomycin that is widely used in tobacco and the like, it is not possible to completely remove non-transformants according to aadA genes/spectinomycin screening.

As an effective transformant screening method for monocot plants, a screening method in which some drug resistance genes are used as selectable markers in transformation whose targets are nuclear genomes has been developed. A screening method using the antibiotic hygromycin and resistance genes thereof is most widely used. A screening method using the herbicide phosphinothricin or glyphosate is also effectively used. However, since a eukaryotic protein expression system of nuclear genomes is different from a protein expression system of plastid genomes including a prokaryotic system, hygromycin that is effective only in a eukaryotic system is not available for a plastid transformation method. Some methods among screening methods using drug resistance genes that are used in transformation whose targets are nuclear genomes can be applied to a plastid transformation method of monocot plants, but effects of such screening methods are limited and unstable (refer to Non-Patent Document 7). In addition, in monocot plant rice, as a screening method in transformation whose targets are nuclear genomes, a nuclear transformation method using nitrite reductase genes as selectable marker genes is reported (refer to Patent Document 2 and Non-Patent Document 8).

On the other hand, among techniques related to a transformation process, as a method of promoting gene introduction into plastids, a method in which exogenous genes are introduced into a callus in which plastids are differentiated and whose regeneration ability is not reduced is disclosed (refer to Patent Document 3). In the method, a green callus induced from transformed rice in which a transcription factor of promoting development of chloroplasts is overexpressed is used as an introduction material. The method showed higher introduction efficiency than a method in which plastids serving as a general introduction material are introduced into an undeveloped yellowish white callus. However, in the method, it is necessary to prepare a strain that appropriately forms a greening callus by performing transformation of nuclear genomes in advance, and applicability to other monocot plants is unknown in addition to an increase in the number of processes

### CITATION LIST

### Patent Literature

### [Patent Document 1]

United States Patent Application, Publication No. 2005/0198704

### [Patent Document 2]

PCT International Publication No. 2005/012520WO

### [Patent Document 3]

Japanese Patent No. 5320782

### Non Patent Literature

### [Non-Patent Document 1]

Svab and Maliga, Proceedings of the National Academy of Sciences of the United States of America, 1993, vol. 90, p. 913-917.

### [Non-Patent Document 2]

Carrer et al., Molecular Genetics and Genomics, 1993, vol. 241, p. 49-56.

### [Non-Patent Document 3]

Hunag et al., Molecular Genetics and Genomics, 2002, vol. 268, p. 19-27.

### [Non-Patent Document 4]

Ye et al., Plant Physiology, 2003, vol. 133, p. 402-410.

### [Non-Patent Document 5]

Khan and Maliga, Nature Biotechnology, 1999, vol. 17, p. 910-915.

### [Non-Patent Document 6]

Lee et al., Molecules and Cells, 2006, vol. 21, p. 401-410.

### [Non-Patent Document 7]

Li et al., Agricultural Sciences in China, 2009, vol. 8, p. 643-651.

### [Non-Patent Document 8]

Nishimura et al., Proceedings of the National Academy of Sciences of the United States of America, 2005, vol. 102, p. 11940-11944.

### SUMMARY OF INVENTION

### Technical Problem

When a nuclear transformation method in which nitrite reductase genes reported in Patent Document 2 and the like are used as selectable marker genes is used, it is possible to prepare nuclear transformants of rice with sufficient screening efficiency. However, it is unknown whether the screening method can be applied for plastid transformation.

The present invention provides a method of producing plastid transformants with high efficiency for a wide range of plant species including monocot plants in which gene introduction has been very difficult so far and screening of transformants using drug resistance has been very difficult.

### Solution to Problem

The inventors have conducted studies to address the above-described problems and found that, when plastid transformants are prepared, genes coding for proteins having a function of neutralizing nitrite toxicity such as nitrite reductase genes are inserted as selectable marker genes, it is possible to remarkably improve screening efficiency, and it is possible to prepare plastid transformants in a wide range of plant species including monocot plants with high efficiency, and completed the present invention.

That is, a method of producing plastid transformants, plastid transformants, and a method of producing proteins according to the present invention include the following aspects.

[1] A method of producing plastid transformants includes a process (a) in which genes coding for proteins having a function of neutralizing nitrite toxicity are introduced into plant tissues; and a process (b) in which the plant tissues into which the genes are introduced in the process (a) are cultured on a medium containing nitrite nitrogen having a concentration at which growth of wild type plant cells is suppressed or a concentration at which regeneration of wild type plants is suppressed, and thus plastid transformants obtained by inserting the genes into plastid genomes are screened.

[2] The method of producing plastid transformants according to Item [1],

wherein the genes are nitrite reductase genes.

[3] The method of producing plastid transformants according to Item [1] or [2],

wherein the plastids include at least one plastid selected from the group consisting of chloroplasts, leucoplasts, amyloplasts, etioplasts, elaioplasts, proteinoplasts, and proplastids.

[4] The method of producing plastid transformants according to any of Items [1] to [3],

wherein the process (a) further includes introducing other exogenous genes of one or two or more species into the plant tissues.

[5] The method of producing plastid transformants according to any of Items [1] to [4],

wherein the process (b) further includes forming plants from the screened plastid transformants.

[6] Plastid transformants in which genes coding for proteins having a function of neutralizing nitrite toxicity are inserted into plastid genomes.

[7] The plastid transformants according to Item [6],

wherein the genes are nitrite reductase genes.

[8] A method of producing proteins further including recovering proteins encoded by the exogenous genes from the plastid transformants produced by the method of producing plastid transformants of Item [4] or plants formed from the plastid transformants.

### Advantageous Effects of Invention

The method of producing plastid transformants according to the present invention has excellent screening efficiency. Therefore, it is possible to produce plastid transformants with high efficiency in both dicotyledonous plants and monocot plants. In particular, a screening method in which genes coding for proteins having a function of neutralizing nitrite toxicity are used as selectable marker genes is very suitable as a screening method for a variety of plants.

### BRIEF DESCRIPTION OF DRAWINGS

FIG. 1 is a pattern diagram of a construct of a switchgrass plastid transformation vector.

FIG. 2A is a diagram showing results of a callus growth rate of switchgrass in different concentrations of sodium nitrite in Example 1<7>.

FIG. 2B is a diagram showing results of a regeneration rate of switchgrass plants in different concentrations of sodium nitrite in Example 1<7>.

FIG. 3 is an electrophoretic pattern of a PCR amplification product obtained in PCR that is performed to confirm whether a transformation vector has been introduced into five individual subcultured plants in Example 1<8>.

### DESCRIPTION OF EMBODIMENTS

A method of producing plastid transformants according to the present invention includes the following processes (a) and (b), a process (a) in which genes coding for proteins having a function of neutralizing nitrite toxicity are introduced into plant tissues, and a process (b) in which the plant tissues into which the genes have been introduced in the process (a) are cultured on a medium containing nitrite nitrogen having a concentration at which growth of wild type plant cells is suppressed or a concentration at which regeneration of wild type plants is suppressed, and thus plastid transformants obtained by inserting the genes into plastid genomes are screened.

Further, as another aspect, in the producing method, the process (a) may further include introducing other exogenous genes of one or two or more species into the plant tissues; and the process (b) may further include forming plants from the screened plastid transformants.

In this specification, the term “plant tissues” refers to plant tissues having plastids or proplastids, and include, for example, differentiated plant tissues, a callus that is differentiated until plastids develop, and an undifferentiated callus.

Examples of the “medium containing nitrite nitrogen having a concentration at which growth of wild type plant cells is suppressed or a concentration at which regeneration of wild type plants is suppressed” include a medium containing sodium nitrite having a concentration of 0.5 to 2.0 g/L, more preferably 0.8 to 2.0 g/L.

The phrases “neutralizes nitrite toxicity,” and “nitrite toxicity neutralization” mean that excess nitrite in plants is metabolized and thus cell toxicity due to nitrite is neutralized.

In the method of producing plastid transformants according to the present invention, “genes coding for proteins having a function of neutralizing nitrite toxicity” (hereinafter referred to as “nitrite toxicity neutralizing genes”) are used as selectable markers. In plants, nitrite is known as an intermediate metabolite in a nitrogen metabolism process performed inside plastids and known to cause cell toxicity. In general, since nitrite is quickly metabolized to ammonia by nitrite reductase, toxic effects are not observed. However, when culture is performed under conditions in which the amount of nitrite exceeds an inherent metabolic capacity of nitrite reductase, callus growth and plant regeneration are significantly suppressed. When the nitrite toxicity neutralizing genes are introduced into plastids according to plastid transformation, excess nitrite in plants is metabolized by proteins having a function of neutralizing nitrite toxicity in transformants. As a result, plant tissues can survive under conditions in which the amount of nitrite exceeds an inherent metabolic capacity of nitrite reductase.

Nitrite reductase genes are preferable as the nitrite toxicity neutralizing genes. The nitrite reductase genes are not particularly limited as long as the genes coding for proteins having a nitrite reductase activity. The nitrite reductase genes may be derived from species of plants serving as hosts or the nitrite reductase genes may be derived from organisms whose species are different from hosts. In addition, the nitrite reductase genes may be genes coding for natural nitrite reductase included in original organisms, or may be genes coding for proteins in which natural nitrite reductase is appropriately modified. Examples of the modified proteins include proteins in which at least one amino acid is deleted, substituted or added without damaging a nitrite reductase activity and proteins in which various tags are added to the N-terminus or the C-terminus of nitrite reductase. Examples of the tag include tags that are widely used in expression and purification of recombinant proteins such as His tag, hemagglutinin (HA) tag, Myc tag, and Flag tag. Also, examples of the genes coding for natural nitrite reductase may include natural nitrite reductase genes and nitrite reductase genes whose degenerate codons are substituted with codons whose codon usage is high in plants serving as hosts.

As the nitrite reductase genes, specifically, genes coding for ferredoxin-nitrite reductase (hereinafter abbreviated as “NiR genes”) are exemplified. The ferredoxin-nitrite reductase is nitrite reductase that uses ferredoxin as an electron donor, and converts nitrite ions into ammonia ions. Examples of the nitrite reductase genes include nitrite reductase genes derived from monocot plants such as NiR genes derived from Oryza sativa (D50556.1, Non-Patent Document 8), NiR genes derived from Zea mays (EU957616.1, XP_008648080.1, Lahners K. et al., Plant Physiol. 1988, vol. 88, p 741-746), NiR genes derived from Triticum (FJ527909.1), NiR genes derived from Hordeum vulgare (AK371794.1, S78730.1), and NiR genes derived from Sorghum bicolor (XM_002454557.1); nitrite reductase genes derived from dicotyledonous plants such as nitrite reductase genes derived from Nicotiana tabacum (AB093533.1, AB245431.1, Kronenberger J. et al., Mol. Gen. Genet., 1993, vol. 236, p. 203-208.), nitrite reductase genes derived from Arabidopsis thaliana (NM_127123.2), nitrite reductase genes derived from Cucumis sativus (EF397679.1), nitrite reductase genes derived from Solanum tuberosum (U76701.1), nitrite reductase genes derived from Medicago polymorpha (XM_003614772.1), nitrite reductase genes derived from Camellia sinensis (JX987133.1), nitrite reductase genes derived from Pyrus calleryana (KC545876.1), nitrite reductase genes derived from Prunus persica (AB061671.1), nitrite reductase genes derived from Selaginella (XM_002987367.1), and nitrite reductase genes derived from Spinacia oleracea (Back E. et al., Mol. Gen. Genet., 1988, vol. 212, p. 20-26.); and nitrite reductase genes derived from algae or hepatics such as nitrite reductase genes derived from Physcomitrella patens (XM_001776973.1), nitrite reductase genes derived from Chlamydomonas (XM_001696735.1), and nitrite reductase genes derived from Chlorella vulgaris (XM_005844460.1) (numbers indicate NCBI accession numbers).

As nitrite reductase genes that are introduced into plant tissues in the process (a), NiR genes derived from Poaceae plants are preferable. For example, genes coding for a polypeptide having amino acid sequences indicated by SEQ ID NO: 1, or genes that have amino acid sequences in which at least one amino acid of amino acid sequences indicated by SEQ ID NO: 1 is deleted, substituted or added and code for a polypeptide having a nitrite reductase activity are exemplified.

In this specification, the phrase “having a nitrite reductase activity” means having an action of reducing a concentration of nitrite in plants to a concentration which can be normal growth.

In addition, as another aspect, the phrase “having a nitrite reductase activity” means that plant cells can grow or plants can be regenerated even when culture is performed on a medium having a concentration of sodium nitrite that is 0.7 g/L or more.

Instead of nitrite reductase genes, genes coding for proteins that decrease a concentration of nitrite in plants or proteins having a function of neutralizing toxicity due to nitrite can be used as the nitrite toxicity neutralizing genes. For example, in plant cells, a path through which nitrite is reduced to a nitric oxide according to nitrate reductase is known. Nitrite oxidase causing nitrate to be formed from nitrite is included in some bacteria. Such enzymes can directly neutralize nitrite toxicity by reducing an abundance of nitrite in the same manner as in the nitrite reductase genes. On the other hand, cell toxicity due to nitrite is speculated to be caused by nitrogen dioxide produced when nitrite reacts with peroxidase and hemoglobin that induces oxidation or nitration of proteins, peroxidation of membrane lipids, mutation of DNA, and the like. Therefore, by regulating an abundance of peroxidase and hemoglobin that react with nitrite or a mechanism thereof, nitrite toxicity can be indirectly neutralized. Therefore, genescoding for proteins that decrease an abundance of peroxidase which reacts with nitrite, and genes coding for proteins having a function of suppressing nitrogen dioxide produced by peroxidase and hemoglobin from inducing oxidation of proteins can also be used as the nitrite toxicity neutralizing genes.

That is, an aspect of the “nitrite toxicity neutralizing gene” is at least one gene selected from the group consisting of genes that directly neutralize cell toxicity due to nitrite and genes that indirectly neutralize cell toxicity due to nitrite.

For example, at least one gene selected from the group consisting of nitrite reductase genes, nitrate reductase genes, nitrite oxidase genes, genes coding for proteins that decrease an abundance of peroxidase which reacts with nitrite, and genes coding for proteins having a function of suppressing nitrogen dioxide produced by peroxidase and hemoglobin from inducing oxidation of proteins are exemplified.

In the method of producing plastid transformants according to the present invention, the process (a) includes introducing nitrite toxicity neutralizing genes used as selectable markers of transformants into plant tissues. Specifically, the process (a) includes introducing vectors incorporating the nitrite toxicity neutralizing genes into plant tissues.

The vectors into which the nitrite toxicity neutralizing genes are incorporated are not particularly limited as long as exogenous genes incorporated into the vectors can be expressed inside plastids in plant tissues. The vectors can be obtained by, for example, the following method. Plasmid pBluescript-based vectors (commercially available from Stratagene), pUC-based vectors or the like are used as a vector backbone. At multicloning sites thereof, adjacent homologous recombination target sequences of two types within plastid genome sequences are cloned. Next, an expression cassette in which DNA having a promoter sequence, a 5′ regulatory region, a polynucleotide of nitrite toxicity neutralizing genes, and DNA having a terminator sequence are arranged from the upstream is incorporated between the homologous recombination target sequences of two types. Accordingly, the vector causing the nitrite toxicity neutralizing genes to be expressed inside plastids is obtained.

In addition, a promoter-less expression cassette in which no promoter sequence is incorporated into an upstream of nitrite toxicity neutralizing genes, and a promoter sequence of endogenous genes in plastid genomes or a sequence downstream thereof is used as one homologous recombination target sequence, and a 5′ regulatory region, a polynucleotide of nitrite toxicity neutralizing genes, a terminator sequence, and homologous recombination target sequences of the other side are sequentially included may be incorporated into the downstream thereof. Therefore, the vector causing the nitrite toxicity neutralizing genes to be expressed inside plastids is obtained. In order to incorporate a polynucleotide into an expression vector, well-known gene recombination techniques can be used and commercial expression vector preparation kits may be used.

In the vector, the promoter that is upstream of the nitrite toxicity neutralizing genes and causes the nitrite toxicity neutralizing genes to be expressed is not particularly limited as long as the promoter serves as a promoter inside plastids. A prokaryotic type promoter is preferable.

Examples of the promoter causing the nitrite toxicity neutralizing genes to be expressed inside plastids include a ribosomal RNA operon promoter (Prrn), a psbA gene promoter (PpsbA) coding for D1 proteins of photosystem II, and an rbcL gene promoter (PrbcL) coding for a large subunit of ribulose-1, 5-diphosphate carboxylase.

In addition, examples of sequences of a 5′ regulatory region added to the upstream of nitrite toxicity neutralizing gene sequences include gene 10 gene 5′ untranslated region (5′-UTR) of coliphage T7, 5′-UTR of the rbcL gene, 5′-UTR of the psbA gene, 5′-UTR of H⁺-ATPaseβ subunit gene (atpB) gene, translation initiation codon downstream sequences (downstream box: DB-box) of the psbA gene, DB-box of the tetanus toxin fragment C (TetC) gene, DB-box of the green fluorescent protein (GFP) gene, and DB-box of the neomycin phosphotransferase type II (NPTII) gene.

In the process (a), in order to introduce a vector into plant tissues, various methods known for those skilled in the art such as a particle bombardment method, a polyethylene glycol method, an electroporation method (electroporation), a liposome method, a cationic liposome method, a calcium phosphate precipitation method, a lipofection method, or a microinjection method can be used.

In the process (a), the plant tissues serving as an introduction material for introducing the nitrite toxicity neutralizing genes are not particularly limited as long as the tissues are derived from plants. For example, it is possible to introduce nitrite toxicity neutralizing genes into an undifferentiated callus that is used as a general introduction material when plant transformants are prepared. The undifferentiated callus can be prepared by a general method.

In the transformation of the process (a), as a material for introducing nitrite toxicity neutralizing genes, the plant tissues having the same level of sensitivity to nitrite nitrogen as wild type plant tissues may be used and a genotype thereof is not particularly limited. That is, the introduction material may be wild type plant tissues or mutant plant tissues that have undergone some kind of genetic modification.

In the transformation of the process (a), as the material of introducing nitrite toxicity neutralizing genes, plant tissues having developed plastids may be used. Plastids are not developed in the undifferentiated callus and remain as undifferentiated proplastids (that is, original plastids). However, when the plant tissues having developed plastids are used as the introduction material, it is possible to improve the efficiency of transformation into plastid genomes.

The plant tissues that are used as the introduction material and have developed plastids may be differentiated plant tissues or may be a callus that is differentiated until plastids are developed. In order to induce differentiation of plastids, culture conditions of the callus can be adjusted or plant hormones can be treated. Examples of the plastids include amyloplasts, chloroplasts, etioplasts, elaioplasts, proteinoplasts, chromoplasts, leucoplasts, and proplastids. In the present invention, at least one plastid selected from the group consisting of chloroplasts, amyloplasts, etioplasts, leucoplasts, and proplastids is preferable, and at least one plastid selected from the group consisting of proplastids and chloroplasts is particularly preferable.

For example, when the plastids are chloroplasts, leaf or stem pieces of plants may be used as the introduction material, chloroplasts differentiation may be induced from a greening callus or a callus of multiple shoots. In the greening callus, proplastids in a callus are induced to be differentiated to chloroplasts. In addition, in the multiple shoots, a callus is differentiated and tissues having a plurality of aggregated shoots are formed. A greening callus or multiple shoots can be induced from a callus when the callus is cultured on a medium whose balance of plant hormones contributing to differentiation and dedifferentiation such as auxin and cytokinin is regulated. For example, the concentration of auxin of a culture medium of a callus is reduced and a concentration of cytokinin is increased, chloroplast formation is induced and a greening callus is obtained. In addition, multiple shoots may be obtained by culturing a callus on a medium containing only cytokinin at a low concentration from the culture medium of the callus.

As the callus in which chloroplast differentiation is induced, a callus obtained by dedifferentiating cells of tissues containing a great amount of chloroplasts of leaves or a callus (a callus derived from non-green tissues) obtained by dedifferentiating cells of non-green tissues such as mature seeds, immature embryos, and roots may be used. The callus can be produced by a general method, for example, culturing plant tissues on a medium containing plant hormones such as auxin and cytokinin.

The plant tissues having developed plastids can be obtained by overexpressing genes coding for proteins that promote differentiation of plastids in the callus. In addition, a callus derived from transformants in which the genes coding for proteins that promote differentiation of plastids are overexpressed may be induced and differentiated to form plastids. Examples of the genes coding for proteins that induce differentiation include GLK genes serving as a transcription factor (Rossini et al., Plant Cell, 2001, vol. 13, p. 1231-1244.) and CES 101 genes of Arabidopsis thaliana (RLK family: Niwa et al., Plant Cell Physioly, 2006, vol. 47, p. 319-331.) (refer to Patent Document 3). The genes coding for proteins that promote differentiation of plastids can be introduced into a callus or plants by a method that is generally used in transformation into plants.

The process (b) includes screening plastid transformed cells (referred to as plastid transformants) obtained by inserting the nitrite toxicity neutralizing genes into plastid genomes by culturing the plant tissues into which the nitrite toxicity neutralizing genes are introduced in the process (a) on a medium containing at least nitrite nitrogen having a concentration at which growth of wild type plant cells is suppressed or a concentration at which regeneration of wild type plants is suppressed.

Further, the process (b) may include forming plants from the screened plastid transformants.

In addition, the concentration at which growth of wild type plant cells is suppressed or the concentration of nitrite nitrogen at which regeneration of wild type plants is suppressed on the medium is preferably 0.5 g/L or more, and more preferably 0.8 g/L or more.

In plants, first, the nitrate nitrogen absorbed from the medium is reduced to nitrite by nitrate reductase in the cytoplasm. The resultant toxic nitrite nitrogen is incorporated into plastids, and is then quickly converted into ammonia by proteins encoded by nitrite toxicity neutralizing genes of nitrite reductase, and is used for cell growth and plant regeneration as a nitrogen source. When plants are cultured on a medium containing excess nitrite, cells containing only wild type plastids died due to toxicity of nitrite that is caused by stagnated and accumulated nitrite metabolites. On the other hand, in cells containing transformed plastids into which an overexpression cassette of nitrite toxicity neutralizing genes is incorporated by plastid transformation, since nitrite metabolism is activated, cell growth and plant regeneration are possible. That is, in the process (b), although the plastid transformants obtained by inserting nitrite toxicity neutralizing genes into plastid genomes can grow or be differentiated, growth of non-transformants is suppressed and ultimately the non-transformants died.

A nitrite metabolic system in which the nitrate nitrogen absorbed from the medium is reduced to nitrite by nitrate reductase and the resulting nitrite nitrogen is then quickly converted into ammonia by proteins that nitrite toxicity neutralizing genes code inside plastids is a metabolic system that almost all plants have (Krapp A. Current Opinion in Plant Biology, 2015, vol. 25, p. 115-122.). That is, the method of producing plastid transformants according to the present invention in which nitrite toxicity neutralizing genes are used as selectable markers can be implemented in various plants. Actually, in many plant species having nitrite reductase genes of NiR genes, nitrate reductase genes are included in many plants, for example, Oryza sativa (X15819.1, Hamat H B et al., Mol. Gen. Genet., 1989, vol. 218, p. 93-98.), Zea mays (NM_001305856.1, Morrison K M. et al., Physiologia Plantarum, 2010, vol. 140, p. 334-341.), Hordeum vulgare (X57844.1, Schnorr K M et al., Mol. Gen. Genet., 1991, vol. 227, p. 411-416.), Sorghum bicolor (XM_002444445.1), Setaria italic (XM_004973598.2), Brachypodium distachyon (XM_003570500.2), Nicotiana tabacum (AB245431.1), Arabidopsis thaliana (J03240.1, Crawford N M. et al., Proc. Natl. Acad. Sci. U.S.A., 1988, vol. 85, p. 5006-5010.), Capsella rubella (XM_006300626.1), Brassica napus (D38220.1), Camelina (XM_010473671.1), castor oil plant (AF314093.1), Beta vulgaris ssp. (EU163265.1), Raphanus sativus var. sativus (KM272859.1), Daucus carota subsp (HQ402930.1), Glycine max (NM_001251221.1, Wu S., et al., Plant Mol. Biol., 1995, vol. 29, p. 491-506.), Phaseolus vulgaris (XM_007141046.1), Fragaria (XM_004300392.2), Medicago polymorpha (HQ840748.1), Solanum lycopersicum (XM_004250307.2), Solanum tuberosum (NM_001288022.1), Cucumis sativus L. (HM755943.1), Cucumis melo (XM_008462231.1), Cucurbita (M33154.1), Spinacia oleracea (M32600.1), Sesamum indicum (XM_011099324.1), Gossypium spp (XM_012612465.1), Nelumbo nucifera (XM_010247609.1), Vitis vinifera (JF796047.1), Amygdalus persica (AB061670.1), Citrus unshiu (XM_006434048.1), Pyrus calleryana (KC545876.1), Theobroma cacao (XM_007018888.1), Elaeis guineensis (XM_010910909.1), Phoenix dactylifera (XM_008796303.1), Jatropha curcas (XM_012222767.1), Populus (XM_011013830.1), Eucalyptus (XM_010065034.1), Betula platyphylla (X54097.1), Tilia platyphyllos (AY138811.1), Morus alba (KF992020.1), Musa acuminate (XM_009419369.1), Physcomitrella patens (AB231618.1), Chlorella (U39930.1), Volvox (XM_002955110.1), and Dunaliella salina (KC108654.1).

Plants can be formed from plastid transformed cells screened in the process (b). Specifically, the screened plastid transformed cells (plastid transformants) are transplanted on a nitrite-containing medium whose balance of hormones promoting regeneration is adjusted, and cultured in a bright place. Thus, plants are formed.

In the method of producing plastid transformants according to the present invention, in the process (a), other exogenous genes of one or two or more species are additionally introduced into the plant tissues in addition to the nitrite toxicity neutralizing genes. Accordingly, it is possible to produce transformants into which the nitrite toxicity neutralizing genes and the other exogenous genes are introduced. For example, when an expression cassette of the other exogenous genes and an expression cassette of the nitrite toxicity neutralizing genes are incorporated into the same vector, it is possible to produce plastid transformants in which both the nitrite toxicity neutralizing genes and the other exogenous genes are incorporated into plastid genomes. The other exogenous genes are not particularly limited, and preferably construct genes, and more preferably construct genes that code proteins useful as a pharmaceutical agent or an industrial source material.

Examples of the exogenous genes include cellulase.

When transformants into which nitrite toxicity neutralizing genes and exogenous genes are introduced are grown and proliferated, transformants in which proteins that the exogenous genes encode are expressed and accumulate inside plastids of the transformants are obtained. Therefore, when proteins that the exogenous genes encode are recovered from the transformants, it is possible to produce certain proteins. The transformants can grow and proliferate in the same manner as host plants before transformation. In addition, proteins can be recovered from the transformants by the same method as when specific proteins are recovered from general plant tissues in consideration of characteristics of proteins. For example, transformants are decomposed and then homogenized, the obtained tissue extract is fractionated by chromatography or the like, and fractions in which target proteins are concentrated can be recovered.

That is, one aspect of the present invention is a method of producing proteins. The method includes a process in which other exogenous genes of one or two or more species are introduced into plant tissues along with genes coding for proteins having a function of neutralizing nitrite toxicity, a process in which plant tissues into which the nitrite toxicity neutralizing genes are introduced are cultured on a medium containing at least nitrite nitrogen having a concentration at which growth of wild type plant cells is suppressed or a concentration at which regeneration of wild type plants is suppressed, and thus plastid transformants obtained by inserting the genes into plastid genomes are screened, a process in which plants are formed from the screened plastid transformants, and a process in which proteins encoded by the exogenous genes are recovered from the plants.

In particular, the method of producing plastid transformants according to the present invention is preferably used to produce plastid transformants of monocot plants whose gene introduction and transformant screening have been very difficult so far. The method is preferably used to produce plastid transformants of Poaceae plants such as rice, sorghum, corn, wheat, barley, oat, pearl barley, switchgrass, Italian ryegrass, perennial ryegrass, timothy grass, Meadow fescue, millet, fixtail millet, sugar cane, and pearl millet. For example, in large biomass plants of fast growing plants such as Poaceae plants, when genes coding for useful proteins are incorporated into plastid genomes, plastid transformants capable of producing useful proteins in a large scale are obtained.

For example, in rice plastid transformants, nitrite reductase genes are inserted into plastid genomes using homologous recombination in plastid genomic DNA of rice, and then cultured on a nitrite-containing medium for screening. Accordingly, the rice plastid transformants can be obtained. As nitrite reductase genes to be inserted into plastid genomes of rice, nitrite reductase genes derived from rice variety Kasalath used in the following example can be used. Homologous recombination in plastid genomic DNA can be performed by a known technique described in Patent Document 3. For example, a vector that is interposed between target homologous recombination regions of plastid genomic DNA of rice and into which nitrite reductase genes are incorporated can be introduced into a callus of rice or a callus in which plastids are differentiated. A position of plastid genomic DNA into which nitrite reductase genes are incorporated is not particularly limited, and is preferably inserted between, for example, trnI genes and trnA genes. In addition, in order to screen transformants into which nitrite reductase genes are incorporated, similarly to Patent Document 2, culture is preferably performed on a medium into which nitrite having a concentration at which growth or regeneration of plants is suppressed in general wild type rice is added for screening.

### EXAMPLES

Next, the present invention will be described in further detail with reference to examples. However, the present invention is not limited to the following examples.

### Example 1

### <1> Preparation of a Switchgrass Regeneration Strain

Skins of mature seeds of switchgrass variety “Alamo” were removed by 60% (v/v) sulfate treatment, and a fungicide for seeds was used for sterilization. The sterilized seeds were further subjected to surface sterilization by hypochlorous acid treatment, and remained on a callus induction medium (a PVC medium: MS salts and vitamins, 30 g/L maltose, 22.6 μM 2, 4-dichlorophenoxyacetic acid, 4.4 μM BAP, 2.5 mM MES, 3 g/L gellan gum, pH 5.7). The seeds were cultured for a few months in the dark at 28° C. Then, grown calluses were classified as independent strains for each derived seed. Strains in which a compact callus expected to have a regeneration ability was grown were selected and cultured. Additionally, compact calluses of the selected strains remained on a regeneration medium (a PVS medium: MS salts and vitamins, 30 g/L maltose, 1.4 μM Gibberellin A₃, 2.5 mM MES, 3 g/L gellan gum, pH 5.7) and were cultured for 4 weeks under light at 28° C., and then a regeneration ability of plants was confirmed. Calluses of strains that were confirmed to have a high regeneration ability were subcultured and grown as experiment materials.

### <2> Screening of Selectable Drugs

In order to develop a new screening method that is appropriate for plastid transformation, the calluses of the regeneration strains prepared in the above <1> were used to examine sensitivity of wild type plant cells to various drugs. Sensitivity of cells to drugs was confirmed according to whether switchgrass calluses were grown when cultured on PVC mediums containing different drug concentrations. Further, for some drugs, calluses remained on the PVS mediums containing different drug concentrations, and a suppression effect of plant regeneration was also confirmed.

Table 1 shows sensitivity examination results. In the table, “A” indicates that callus growth and plant regeneration were suppressed, “B” indicates that callus growth and plant regeneration were not suppressed, and “-” indicates that no experiment was performed.

TABLE 1

| | Callus growth | | | | Shoot regeneration | | | |
|---|---|---|---|---|---|---|---|---|
| | | | Growth | | | | Regeneration | |
| | Experiment | | supression | | Experiment | | supression | |
| | concentration | | (supression | | concentration | | (supression | |
| Drugs | range | | concentration) | | range | | concentration) | |
| Sectinomycin | 0 to 800 | mg/L | B | | 0 to 800 | mg/L | B | |
| Streptomycin | 0 to 800 | mg/L | B | | 0 to 800 | mg/L | A | (>400 mg/L) |
| G418 | 0 to 100 | mg/L | A | (>25 mg/L) | 0 to 100 | mg/L | A | (>25 mg/L) |
| Kanamycin | 0 to 200 | mg/L | A | (>200 mg/L) | 0 to 300 | mg/L | A | (>100 mg/L) |
| Gentamicin | 0 to 100 | mg/L | B | | — | | — | |
| Neomycin | 0 to 100 | mg/L | B | | — | | — | |
| Ribostamycin | 0 to 100 | mg/L | B | | — | | — | |
| Paromomycin | 0 to 100 | mg/L | B | | — | | — | |
| Cloramphenicol | 0 to 45 | mg/L | A | (>30 mg/L) | 0 to 50 | mg/L | A | (>15 mg/L) |
| Tetracycline | 0 to 60 | mg/L | A | (>40 mg/L) | 0 to 100 | mg/L | A | (>60 mg/L) |
| Troleandomycin | 0 to 200 | mg/L | B | | 0 to 200 | mg/L | B | |
| Erythromycin | 0 to 200 | mg/L | B | | 0 to 200 | mg/L | B | |
| Bleocin | 0 to 75 | mg/L | A | (>25 mg/L) | 0 to 25 | mg/L | A | (>10 mg/L) |
| Zeocin | 0 to 1000 | mg/L | B | | 0 to 10 | mg/L | B | |
| Bleomycin | 0 to 30 | mg/L | B | | — | | — | |
| Puromycin | 0 to 100 | mg/L | B | | — | | — | |
| Glyphosate | 0 to 2 | mM | A | (>0.1 mM) | 0 to 2 | mM | A | (>0.1 mM) |
| Fluazifop-butyl | 0 to 10 | μM | A | (>1.0 μM) | 0 to 10 | μM | A | (>0.1 μM) |
| Cycloxydim | 0 to 10 | μM | A | (>1.5 μM) | 0 to 2 | μM | A | (>0.1 μM) |
| Clethodim | 0 to 2 | μM | B | | — | | — | |
| Clodinafop | 0 to 2 | μM | A | (>1.0 μM) | — | | — | |
| Sethoxydim | 0 to 2 | μM | B | | — | | — | |
| Haloxyfop | 0 to 2 | μM | A | (>1.0 μM) | — | | — | |
| Fosfomycin | 0 to 75 | μM | B | | — | | — | |
| Pinoxaden | 0 to 2 | μM | A | (>1.0 μM) | — | | — | |
| Methylviologen | 0 to 50 | μM | A | (>1.0 μM) | 0 to 7 | μM | A | (>1.0 μM) |
| Butafenacil | 0 to 0.5 | μM | B | | — | | — | |
| Sodium nitrite | 0 to 1000 | μM | A | (>800 mg/L) | 0 to 1000 | mg/L | A | (>800 mg/L) |

<3> Preparation of Plastid Transformation Vectors in which Various Drug Resistance Genes were Used as Selectable Markers.

Vectors for plastid transformation in which genes having resistance to kanamycin, chloramphenicol, bleomycin, cycloxydim, fluazifop-butyl, and sodium nitrite among drugs in which a suppression action of normal cell proliferation was confirmed in the above <2> were used as selectable marker genes were prepared. Specifically, AphA-6 (kanamycin resistance genes), CAT (chloramphenicol resistance genes, Ble (bleomycin resistance genes), mCT (cycloxydim and fluazifop-butyl resistance genes: mutant CT domain of ACCase enzymes), and PSR1 (a region excluding a signal peptide region of 5′ terminal from cDNA of nitrite reductase genes derived from rice variety Kasalath) (SEQ ID NO: 2) were used as selectable marker genes. The selectable marker genes that were isolated or artificially synthesized by PCR were used. A region in which homologous recombination with plastid genomes occurred, a terminator and a translational control sequence were selected and designed with reference to high expression examples of exogenous proteins that have been reported so far (Daniell et al., Methods in Molecular Biology, 2005, Vol. 286, p. 111-138; Verma and Daniell, Plant Physiology, 2007, Vol. 145, p. 1129-1143). Component sequences were amplified by PCR and linked using restriction enzyme sites, or subjected to an InFusion method (Clontech), and thus vectors were prepared.

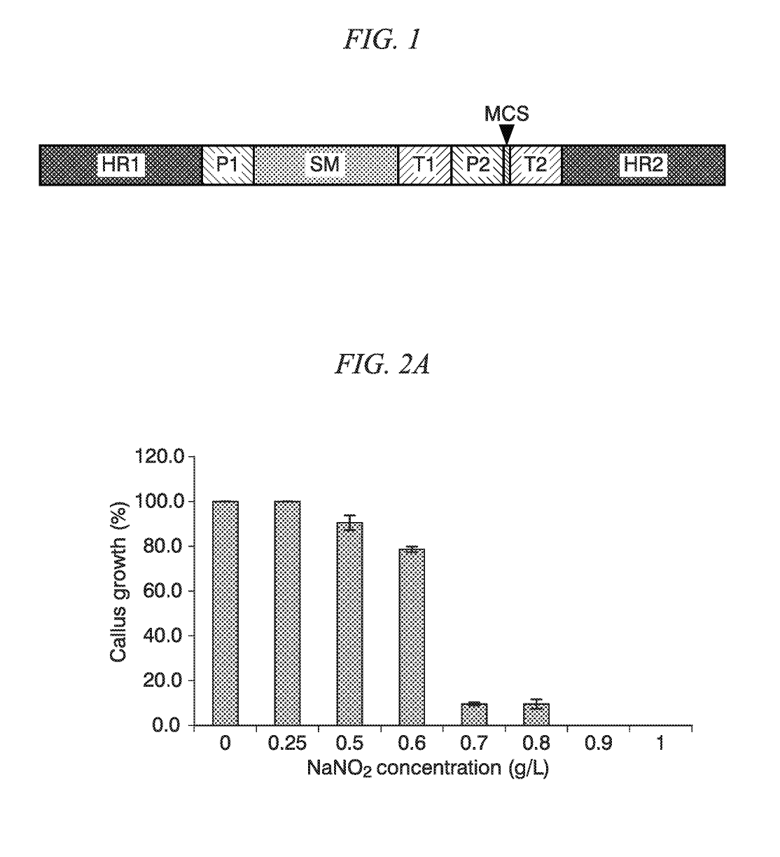

FIG. 1 shows a construct of a plastid transformation vector. In FIG. 1, “HR1” indicates a plastid genome homologous recombination sequence (trnI region) (SEQ ID NO: 3), “HR2” indicates a plastid genome homologous recombination sequence (trnA region) (SEQ ID NO: 4), “P1” indicates a promoter (rrn promoter) and a translational control sequence (T7g10) linked to a downstream thereof, “SM” indicates selectable marker genes, “T1” indicates a terminator (psbA terminator), “P2” indicates a promoter (psbA promoter), “MCS” indicates a multcloning site serving as an insertion site of certain genes, and “T2” indicates a terminator (rps16 terminator).

### <4> Formation of a Greening Callus and Multiple Shoots

A greening callus and multiple shoots were formed in the switchgrass regeneration strain prepared in the above <1>.

Specifically, among calluses prepared and subcultured in the above <1>, calluses that were cultured for 3 to 4 weeks after subculture remained on a greening and multiple shoot induction medium (ML1R3 medium: Ahmadabadi et al., (2007) Transgenic Research, vol. 16, p. 437-448), and cultured for about 4 weeks under light at 28° C., and thus a greening callus and multiple shoots were obtained.

<5> Gene Introduction into Switchgrass Plastids and Screening of Transformed Cells

The plastid transformation vector prepared in the above <3> was introduced into the switchgrass greening callus and multiple shoots and transformant cells were screened. As a control group for comparison of introduction efficiency, a subculture callus before greening induction was used.

First, the switchgrass greening callus and multiple shoots prepared in the above <4> were chopped with a scalpel to tissue pieces of about 2 mm, and arranged in a circle having a diameter of 35 mm in a central part of the PVC medium. Separately, the plastid transformation vectors prepared in the above <3> were purified by a Hispeed Plasmid Midi Kit (commercially available from QIAGEN) and coated with gold particles of 0.6 μm. The plastid transformation vectors coated with the gold particles were introduced into the tissue pieces arranged in the PVC medium by particle bombardment. Adjustment of a DNA-gold particle solution and gene introduction by particle bombardment were performed according to a method of Okuzaki et al (Okuzaki and Tabei, Plant Biotechnology, 2012, vol. 29, p. 307-310).

The tissues after introduction treatment were spread across several PVC media, and cultured for 4 to 7 days in the dark at 28° C. The tissues after introduction and culture were additionally cultured under screening conditions of Table 2 for each type of the plastid transformation vector, and transformed plastids and cells containing transformed plastids were screened. The tissues after screening culture remained on PVS media containing various selectable drugs and plant regeneration was induced under light at 28° C.

TABLE 2

| Seletable marker | Selectable | Selectable drug | |
|---|---|---|---|
| genes | drugs | concentration | |
| AphA-6 | Kanamycin | 100 to 200 | mg/L |
| CAT | Chloramphenicol | 15 to 30 | mg/L |
| Ble | Bleomycin | 5 to 75 | mg/L |
| mCT | Cycloxydim | 0.5 to 2.0 | μM |
| | Fluazifop-butyl | | |
| PSR1 | Sodium nitrite | 800 to 1000 | mg/L |

### <6> Confirmation of Gene Introduction

After the screening culture, tissues of 2 mm piece were sampled from regenerated plants. According to the presence of amplified DNA fragments using a primer set that specifically amplifies transformation vector fragments by Phire Plant Direct PCR Kit (commercially available from Thermo Fisher Scientific), it was confirmed whether the transformation vector was introduced. After recovery and purification, sequences of the amplified DNA fragments were confirmed and the fragments were confirmed as target DNA fragments. These plants were confirmed as target plastid transformants.

In addition, Table 3 shows the number of plates of plate media on which an introduction experiment was performed by various screening methods and the number of transformant strains in which introduction of a transformation vector was confirmed. Without sodium nitrite screening, in all plants that were regenerated after screening, amplified DNA fragments derived from the transformation vector were not confirmed and no plastid transformants were obtained. On the other hand, in the sodium nitrite screening, plastid transformants of 15 strains were obtained from 48 plates by transformation to a callus before greening induction, and plastid transformants of 5 strains were obtained from 12 plates by transformation to a greening callus and multiple shoots. Based on such results, it was found that sodium nitrite screening in which nitrite reductase genes were used as selectable markers was effective in acquiring plastid transformants, and particularly, chloroplast transformants were very efficiently obtained by performing introduction into a greening callus and multiple shoots.

TABLE 3

| | | | The number of |
|---|---|---|---|
| | | The number of | gene |
| | | introduction | introduction |
| Seletable | Introduction | experiment | confirmation |
| drugs | tissues | plates | strains |
| Kanamycin | Callus | 11 | 0 |
| | Greening callus | 16 | 0 |
| | and multiple shoots | | |
| Chloramphenicol | Callus | 39 | 0 |
| | Greening callus | 53 | 0 |
| | and multiple shoots | | |
| Bleomycin | Callus | 24 | 0 |
| | Greening callus | 4 | 0 |
| | and multiple shoots | | |
| Cycloxydim, | Callus | 24 | 0 |
| fluazifop-butyl | Greening callus | 10 | 0 |
| | and multiple shoots | | |
| Sodium nitrite | Callus | 48 | 15 |
| | Greening callus | 12 | 5 |
| | and multiple shoots | | |

### <7> Study of Detailed Conditions of Sodium Nitrite Screening

In the sodium nitrite screening, in order to determine a screening concentration more effectively, wild type switchgrasses were cultured under sodium nitrite conditions of different concentrations, and the sensitivity of wild type cells to sodium nitrite was examined.

Specifically, a mass of 14 switchgrass calluses remained for each plate on PVC media and PVS media containing sodium nitrite having a concentration of 0, 0.25, 0.5, 0.6, 0.7, 0.8, 0.9, or 1.0 g/L, and three plates of each of which were prepared for each concentration condition. The PVC media were cultured in the dark at 28° C. and the PVS media were cultured under light at 28° C. for 4 weeks. After culture, the number of grown calluses was measured in the PVC media, and the number of calluses from which plants were regenerated was measured in the PVS media. The growth rate and the regeneration rate were calculated.

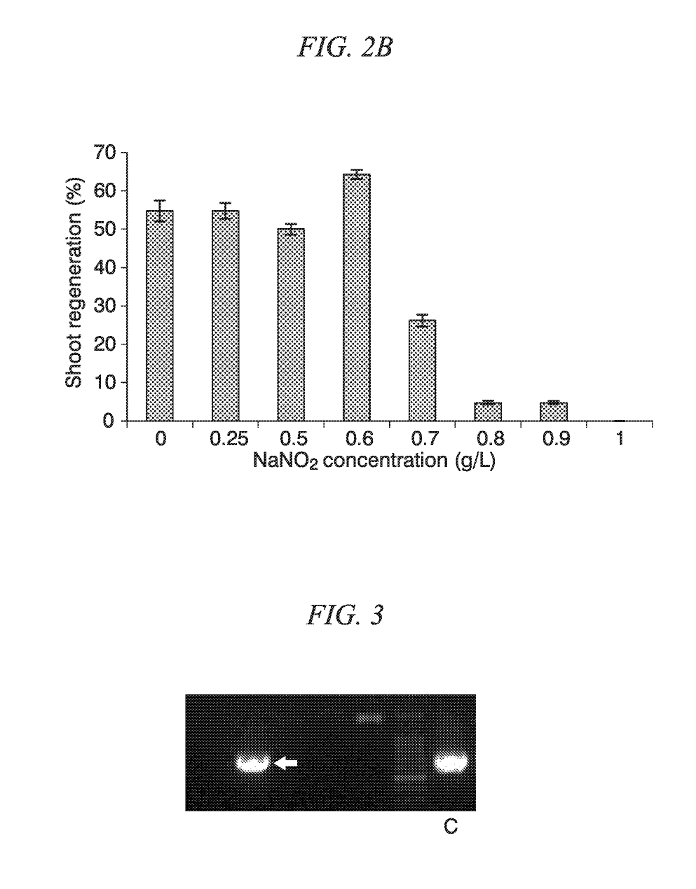

FIG. 2A shows results of a callus growth rate in different concentrations of sodium nitrite. FIG. 2B shows results of a plant regeneration rate in different concentrations of sodium nitrite. The callus growth in the PVC media was sharply reduced in sodium nitrite at 0.7 g/L and completely suppressed at 0.9 g/L or more (FIG. 2A). On the other hand, plant regeneration in the PVS media was sharply reduced in sodium nitrite at 0.8 g/L and completely suppressed at 1.0 g/L (FIG. 2B). Such results indicate that a concentration of sodium nitrite in screening of transformed switchgrass cells was preferably 0.8 g/L or more.

### <8> Maintaining Insertion Genes Obtained by Sodium Nitrite Screening

The regenerated plants that were screened by sodium nitrite screening and in which amplification of insertion gene fragments by PCR performed in the above <6> was confirmed were transplanted and started to root on an MS medium (½ MS salts and vitamins, 15 g/L sucrose, 2.5 mM MES, 3 g/L gellan gum, pH 5.7) containing sodium nitrite having a concentration of 0.8 g/L. Then, the sample was subcultured on a new MS medium containing sodium nitrite having a concentration of 0.8 g/L every 1 to 2 months. Tillers generated during subculture were separated and subcultured according to the same strain. After about 1 year, PCR was performed on five individual plants from the strains in the same manner as in the above <6>. As a result, amplified DNA fragments were confirmed in one individual, and stably maintained insertion genes were confirmed. FIG. 3 shows the electrophoresis result of the PCR amplification product. In FIG. 3, a band marked with an arrow indicates amplified DNA fragments, and “C” indicates a lane in which a positive control (a PCR product obtained using a plastid transformation vector as a template) flowed.

### Example 2

<1> Gene Introduction into Rice Plastids and Screening of Transformed Cells

PSR1 genes were introduced into plastids of rice variety Nipponbare and rice variety Koshihikari and transformants were screened using sodium nitrite. Specifically, it was performed as follows. An MS-NK medium (Nishimura et al., Nature Protocols, 2006, Vol. 1, p. 2796-2802) used as a regeneration medium contained nitrate nitrogen, originally. A wild type of rice variety Nipponbare had a regeneration ability in an MS-NK medium containing sodium nitrite having a concentration of 400 mg/L. However, a wild type of rice variety Koshihikari was not regenerated even in an MS-NK medium in which nitrite reductase was weak and no sodium nitrite was added.

Calluses derived from blastocysts according to culture of mature rice seeds were induced and prepared using a method of the related art. The obtained calluses of about 2 mm were arranged in a circle having a diameter of about 35 mm in a central part of an N6D medium (Nishimura et al., Nature Protocols, 2006, vol. 1, p. 2796-2802) and used as an introduction material. In the same manner as in the switchgrass of Example 1<5>, a DNA-gold particle solution was prepared and introduced into the calluses. The calluses after the introduction treatment were spread across several N6D media and cultured for 3 to 9 days in the dark at 29.5° C. Then, calluses derived from rice variety Nipponbare were transplanted to an MS-NK medium containing sodium nitrite having a concentration of 800 to 1000 mg/L and calluses derived from rice variety Koshihikari were transplanted to an MS-NK medium containing sodium nitrite having a concentration of 0 to 400 mg/L, and plant regeneration was induced in a bright place at 29.5° C. Tissue pieces of about 2 mm were sampled from the obtained regenerated individuals, and insertion of target DNA fragments was confirmed by PCR in the same manner as in the switchgrass.

TABLE 4

| | The number of | The number of gene |
|---|---|---|
| Introduction | introduction experiment | introduction confirmation |
| varieties | plates | strains |
| Nipponbare | 1 | 38 |
| Koshihikari | 2 | 49 |

Table 4 shows the number of plates of plate media on which an introduction experiment was performed and the number of transformant strains in which introduction of a transformation vector was confirmed. In both varieties, genes were introduced with higher efficiency than in the switchgrass.

### INDUSTRIAL APPLICABILITY

The method of producing plastid transformants has an excellent screening efficiency, and can produce plastid transformants with high efficiency in both dicotyledonous plants and monocot plants, and is therefore extremely useful industrially.

1. A method of producing plastid transformants comprising:
a process (a) in which genes coding for proteins having a function of neutralizing nitrite toxicity are introduced into plant tissues; and
a process (b) in which the plant tissues into which the genes are inserted in the process (a) are cultured in a medium containing nitrite nitrogen having a concentration at which growth of wild type plant cells is suppressed or a concentration at which regeneration of wild type plants is suppressed, and thus plastid transformants obtained by inserting the genes into plastid genomes are screened.

2. The method of producing plastid transformants according to claim 1,
wherein the genes are nitrite reductase genes.

3. The method of producing plastid transformants according to claim 1,
wherein the plastids include at least one plastid selected from the group consisting of chloroplasts, leucoplasts, amyloplasts, etioplasts, elaioplasts, proteinoplasts, and proplastids.

4. The method of producing plastid transformants according to claim 1,
wherein the process (a) further includes introducing other exogenous genes of one or two or more species into the plant tissues.

5. The method of producing plastid transformants according to claim 1,
wherein the process (b) further includes forming plants from the screened plastid transformants.

6. Plastid transformants in which genes coding for proteins having a function of neutralizing nitrite toxicity are inserted into plastid genomes.

7. The plastid transformants according to claim 6,
wherein the genes are nitrite reductase genes.

8. A method of producing proteins comprising recovering proteins encoded by exogenous genes from the plastid transformants produced by the method of producing plastid transformants of claim 4 or plants formed from the plastid transformants.

9. The method of producing plastid transformants according to claim 2,
wherein the process (a) further includes introducing other exogenous genes of one or two or more species into the plant tissues.

10. The method of producing plastid transformants according to claim 3,
wherein the process (a) further includes introducing other exogenous genes of one or two or more species into the plant tissues.

11. The method of producing plastid transformants according claim 2,
wherein the process (b) further includes forming plants from the screened plastid transformants.

12. The method of producing plastid transformants according claim 3,
wherein the process (b) further includes forming plants from the screened plastid transformants.

13. The method of producing plastid transformants according claim 4,
wherein the process (b) further includes forming plants from the screened plastid transformants.

14. The method of producing plastid transformants according claim 9,
wherein the process (b) further includes forming plants from the screened plastid transformants.

15. The method of producing plastid transformants according claim 10,
wherein the process (b) further includes forming plants from the screened plastid transformants.

16. A method of producing proteins comprising recovering proteins encoded by exogenous genes from the plastid transformants produced by the method of producing plastid transformants of claim 9 or plants formed from the plastid transformants.

17. A method of producing proteins comprising recovering proteins encoded by exogenous genes from the plastid transformants produced by the method of producing plastid transformants of claim 10 or plants formed from the plastid transformants.


SEQUENCE LISTING (4 sequences)

SEQ ID NO: 1 - protein, 567 aa, Oryza sativa
MPVQSSTVSAPSSSTPAADEAVSAERLEPRVEQREGRYWVLKEKYRTGLNPQEKVKLGKEPMSLFMEGGIKELAKMPMEEIEADKLSKEDIDVRLKWLGLFHRRKHQYGRFMMRLKLPNGVTTSEQTRYLASVIEAYGKEGCADVTTRQNWQIRGVTLPDVPAILDGLNAVGLTSLQSGMDNVRNPVGNPLAGIDPDEIVDTRSYTNLLSSYITSNFQGNPTITNLPRKWNVCVIGSHDLYEHPHINDLAYMPAVKGGKFGFNLLVGGFISPKRWEEALPLDAWVPGDDIIPVCKAVLEAYRDLGTRGNRQKTRMMWLIDELGMEAFRSEVEKRMPNGVLERAAPDDLIDKKWQRRDYLGVHPQKQEGMSYVGLHVPVGRVQAADMFELARLADEYGSGELRLTVEQNIVIPNVKNEKVEALLAEPLLQKFSPQPSLLLKGLVACTGNQFCGQAIIETKQRALLVTSQVEKLVSVPRAVRMHWTGCPNSCGQVQVADIGFMGCLTKDSAGKIVEAADIFVGGRVGSDSHLAGAYKKSVPCDELAPIVADILVERFGAVRREREEDEE

SEQ ID NO: 2 - DNA, 1704 bp, Oryza sativa
atgcccgtgcagtcgtcgacggtgtccgcaccgtcctcctcgactccggcggcggacgaggccgtgtcggcggagcggctggagccgcgggtggagcagcgggagggccggtactgggtgctcaaggagaagtaccggacggggctgaacccgcaggagaaggtgaagctggggaaggagcccatgtcattgttcatggagggcggcatcaaggagctcgccaagatgcccatggaggagatcgaggccgacaagctctccaaggaggacatcgacgtgcggctcaagtggctcggcctcttccaccgccgcaaacatcagtatgggcggttcatgatgcggctgaagctgccaaacggtgtgacgacgagcgagcagacgaggtacctggcgagcgtgatcgaggcgtacggcaaggagggctgcgccgacgtgacaacccgccagaactggcagatccgcggcgtcacgctccccgacgtgccggccatcctcgacgggctcaacgccgtcggcctcaccagcctccagagcggcatggacaacgtccgcaaccccgtcggcaacccgctcgccggcatcgaccccgacgagatcgtcgacacgcgatcctacaccaacctcctctcctcctacatcaccagcaacttccagggcaaccccaccatcaccaacctgccgaggaagtggaacgtgtgcgtgatcgggtcgcacgatctgtacgagcacccgcacatcaacgacctcgcgtacatgccggcggtgaagggcggcaagttcgggttcaacctccttgtcggcgggttcatcagccccaagaggtgggaggaggcgctgccgctggacgcctgggtccccggcgacgacatcatcccggtgtgcaaggccgttctcgaggcgtaccgcgacctcggcaccaggggcaaccgccagaagacccgcatgatgtggctcatcgacgaacttggaatggaggcttttcggtcggaggtggagaagaggatgccgaacggcgtgctggagcgcgctgcgccggacgacctcatcgacaagaaatggcagaggagggactacctcggcgtgcacccgcagaagcaggaagggatgtcctacgtcggcctgcacgtgcccgtcggccgggtgcaggcggcggacatgttcgagctcgcccgccttgccgacgagtatggctccggcgagctccgcctcaccgtggagcagaacatcgtgatcccgaacgtcaagaacgagaaggtggaggcgctgctcgccgagccgctgcttcagaagttctccccgcagccgtcgctgctgctcaagggcctggtcgcgtgcaccggcaaccagttctgcggccaggccatcatcgagacgaagcagcgggcgctgctggtgacgtcgcaggtggagaagctcgtgtcggtgccccgggcggtgcggatgcactggaccggctgccccaacagctgcggccaggtgcaggtcgccgacatcggcttcatgggctgcctcaccaaggatagcgccggcaagatcgtcgaggcggccgacatcttcgtcggcggccgcgtcggcagcgactcgcacctcgccggcgcgtacaagaagtccgtgccgtgcgacgagctggcgccgatcgtcgccgacatcctggtcgagcggttcggggccgtgcggagggagagggaggaggacgaggagtag

SEQ ID NO: 3 - DNA, 2127 bp, Panicum virgatum
gcactctgctgggccgacactgacactgagagacgaaagctaggggagcaaatgggattagagaccccagtagtcctagccgtaaacgatggatactaggtgctgtgcgactcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccctcgggaacgcggacacaggtggtgcatggctgtcgtcagctcgtgccgtaaggtgttgggttaagtctcgcaacgagcgcaaccctcgtgtttagttgccactataagtttggaaccctgaacagaccgccggtgttaagccggaggaaggagaggatgaggccaagtcatcatgccccttatgccctgggcgacacacgtgctacaatgggcgggacaaagggtcgcgatctcgcgagggtgagctaactccaaaaacccgtcctcagttcggattgcaggctgcaactcgcctgcatgaagcaggaatcgctagtaatcgccggtcagccatacggcggtgaatccgttcccgggccttgtacacaccgcccgtcacactataggagctggccgggtttgaagtcattacccttaaccgtaaggagggggatgcctaaggctaggcttgcgactggagtgaagtcgtaacaaggtagccgtactggaaggtgcggctggatcacctccttttcagggagagctaatgcttatgcttattgggtattttggtttgacattgcttcacgcccaaaaacaaaaagaaggcagctacgtctgaactaaacttggatatggaagtcttctttcgtttagggtgaagtaagaccaagctcatgagcttattatcctaggtcggaacaaattagttgatagtgataggatcccctttttgacgtccccatgtccccccgtgtggcggcatggggatgtcaaaaggaaagggatggagtttttctcgcttttggcgtagcaggcctccctttgggaggcccgcgcgacgggctattagctcagtggtagagcgcgcccctgataattgcgtcgttgtgcctgggctgtgagggctctcagccacatggatagttcaatgtgctcatcagcgcctgacccgaagatgtggatcatccaaggcacattagcatggcgtactcctcctgtttgaatcggagtttgaaaccaaacaaacttctcctcaggaggatagatggggcgattcaggtgagatcccatgtagatctaactttctattcactcgtgggatccgggcggtccgggggggggccaccgcggctcctctcttctcgagaatccatacatcccttatcagtgtatggagagctatctctcgagcacaggttgaggttcgtcctcaatgggaaaatggagcacctaacaacgcatcttcacagaccaagaactacgagatcacccctttcattctggggtgacggagggatcgtaccattcgagcctttttttcatgcttttcccggcggtctggagaaagcagtaatcaataggacttccctaatcctcccttcctgaaaggaagaacgtgaaattctttttcctttctgcagggaccaggagattggatctagccataagaggaatgcttggtataaataagccacttcttggtcttcgaccccctaagtcactacgagcgcccccgatcagtgcaatgggatgtggctatttatctatctcttgactcgaaatgggagcagagcaggtttgaaaaaggatcttagagtgtctagggttgggccaggagggtctcttaaccccttcctttttctgcccatcggagttatttcccaaggacttgccatggtaagggggagaagggggaagaagcacacttgaagagcgcagtacaacggggagttgtatgctgcgttcgggaaggatgaatcgctcccgaaaaggagtctattgattctctcccaattggttggatcgtaggggcgatgatttacttcacgggcgaggtctctggtt

SEQ ID NO: 4 - DNA, 2071 bp, Panicum virgatum
cagctgcgccagggaaaagaatagaagaagcatctgactctttcatgcatactccacttggctcgggggggatatagctcagttggtagagctccgctcttgcaattgggtcgttgcgattacgggttggctgtctaattgtccaggcggtaatgatagtatcttgtacctgaaccggtggctcactttttctaagtaatggggaagaggactgaaacatgccactgaaagactctactgagacaaaaagatgggctgtcaaaaaggtagaggaggtaggatgggcagttggtcagatctagtatggatcgtacatggacgatagttggagtcggcggctctcctaggcttccctcatctgggatccctggggaagaggatcaagttggcccttgcgaatagcttgatgcactatctcccttcaaccctttgagcgaaatgtggcaaaaggaaggaaaatccatggaccgaccccattgtctccaccccgtaggaactacgagatcaccccaaggacgccttcggcgtccaggggtcacggaccgaccatagaccctgttcaataagtggaacacattagccgtccgctctccggttgggcagtaagggtcggagaagggcaatcactcgttcttaaaaccagcattcttaagttaagatcaaagagtcgggcggaaaaaggggagagctccccgttcctggttctcctgtagctggattccccggaaccacaagaatccttagaatgggattccaactcagcaccttttgttttgagattttgagaagagttgctctttggagagcacagtacgatgaaagttgtaagctgtgttcgggggggagttattgcctatcgttgtcctctatggtagaacccgtcggggaggcctgagaggcggtggtttaccctgtggcggatgtcagcggttcgagtccgcttatctccagcccgtgaacttagcggatactgtgatagcaccaattttgccaattcgtcagttcgatctatgatttcgcattcatggacgttgataagatccttccatttagtagcaccttaggatggcatagccttaacgttaatggcgaggttcaaaagaggaaaggcttgcggtggatacctaggcacccagagacgaggaagggcgtagcaagcgacgaaatgcttcggggagttgaaaataagcatagatccggagattcccaaataggtcaaccttttgaactgcctgctgaatccatgagcaggcaagagacaacctggcgaactgaaacatcttagtagccagaggaaaagaaagcaaaagcgattcccgtagtagcggcgagcgaaatgggagcagcctaaaccgtgaaaacggggttgtgggagagcaatacaagcgttgtgctgctaggcgaagcggttgagtgccgcaccctagatggctaaagtccagtagccgaaagcatcactagcttacgctctgacccgagtagcatggggcacgtggaatcccgtgtgaatcagcaaggaccaccttgcaaggctaaatactcctgggtgaccgatagcgaagtagtaccgtgagggaaaggtgaaaagaacccccagtgggtagtgaaatagaacgtgaaaccgtgctgagctcccaagcagtgggaggggaaagtgatctctgaccgcgtgcctgttgaagaatgagccggcgactcataggcagtggcttggttaagggaatggaacccaccggagccgtagcgaaagcgagtcttcatagggcgattgtcactgcttatggacccgaacctgggtgatctatccatgaccaggatgaagcttggatgaaactaagcagaggtccgaaccgactgatgttgaagaatcagcggatgagttgtggttaggggtgaaatgccactcgaacccagagctagctggttctccccgaaatgcgttgaggcgcagcagttgactggacatctaggggtaaagcactgtttcggtgcgggctgcgcgagcggtaccaaatcgaggcaaactctgaatactagatatgacccaaacaggggtcaaggtcggccagtg